(19) United States
(12) Patent Application Publication
(10) Pub. No.: US 2006/0258697 A1
(43) Pub. Date: Nov. 16, 2006

(54) Method of augmenting the antitumor activity of anticancer agents

(76) Inventors: Youcef M. Rustum, Amherst, NY (US); Shousong Cao, East Amherst, NY (US); Farukh Durrani, Getzville, NY (US); Marwan Fakih, Williamsville, NY (US)

Correspondence Address: HODGSON RUSS LLP, ONE M & T PLAZA, SUITE 2000, BUFFALO, NY, 14203-2391, US

(21) Appl. No.: 11/405,377

(22) Filed: Apr. 17, 2006

Related U.S. Application Data
(63) Continuation-in-part of application No. 11/079,633, filed on Mar. 11, 2005, now abandoned.
Application No. 11/079,633 is a continuation-in-part of application No. 10/844,800, filed on May 13, 2004, now abandoned.
(60) Provisional application No. 60/471,183, filed on May 13, 2003.

(51) Int. Cl. A61K 31/198; A61K 31/4745; A61K 31/337

(52) U.S. Cl. 514/283; 514/449; 514/561

(57) ABSTRACT

A method for augmenting the antitumor activity of anti-cancer agents is provided. The method comprises administering to an individual an anti-cancer and a selenium compound. A method is also provided for inhibiting the growth of a tumor which-has proven to be refractory to anticancer agents. The methods comprises administration of selenium compound followed by administration of the anticancer agent.

This application is a continuation-in-part of U.S. application Ser. No. 11/079,633, filed on Mar. 11, 2005, which is a continuation-in-part of U.S. application Ser. No. 10/844,800, filed on May 13, 2004, which in turn claims priority to U.S. provisional application no. 60/471,183 filed on May 13, 2003, the disclosures of which are incorporated herein by reference.

### FIELD OF THE INVENTION

This invention relates generally to the field of cancer therapy and more particularly to a method for augmenting the antitumor activity of chemotherapeutic agents.

### DESCRIPTION OF RELATED ART

Chemotherapy is now a recognized and widely used modality of cancer treatment. Depending upon the type of cancer, chemotherapy is often the primary course of treatment. For example, chemotherapy is widely used either alone or in combination with other treatments such as radiation treatment for a variety of cancers including cancer of the ovary, testis, breast, bladder, colon, head and neck as well as leukemia, lymphomas, sarcomas, melanomas, myelomas and others.

Chemotherapeutic agents are broadly classified into a number of groups. The majority of anticancer drugs act as cytotoxic drugs. The classification of these drugs into groups is mechanism based. While chemotherapeutic agents have proven extremely useful in the treatment of cancer, nearly all of them are associated with significant toxic effects because of their potential to kill cancerous as well as healthy cells. The toxicity associated with anticancer drugs often forces discontinuation of treatment which may negatively impact the prognosis of patient's condition and clinical outcome and result in compromising the quality of life.

In the field of cancer therapy there is an ongoing need to identify new chemotherapeutic agents or to increase the potency of existing agents. While some recent in vitro studies have attempted to address the issue of toxicity of anticancer agents by selenium compounds (Steifel et al., 1999, WO 99/64018; Chen et al., 1986, J. Nutrition, 116(12):2453-2465; Dobric et al., 1998, J. Environ. Pathol. Toxicol Oncol., 17:291-299, the effects of selenium on the antitumor activity of the chemotherapeutic agents, if any, are not clear.

### SUMMARY OF THE INVENTION

In the present invention it was observed that administration of selenium compounds augments the antitumor activity of anticancer agents. Data is presented for in vivo studies in xenograft bearing animals.

Additionally, clinical studies have been carried out which further confirm augmentation of antitumor activity of anticancer agents by administration of selenium. While tumor shrinkage was observed for several chemotherapeutic agents, the effect was not durable with all chemotherapeutic agents and complete tumor regression (cure) was observed only with irinotecan and taxotere.

Further, it was observed that the augmentation of activity was provided by the administration of seleno-L-methionine (SLM) and methylseleno cysteine (MSC), but not sodium selenite (SS) or methylseleninic acid (MSA). The selenium compound is preferably administered at least three days prior to the administration of the anti-tumor agent.

Accordingly, the present invention discloses a method for augmenting the antitumor activity of anticancer agents selected from the group consisting of irinotecan and taxotere. The method comprises administering to an individual having a tumor, the anti-tumor agent and a selenium compound selected from the group consisting of SLM and MSC. In one embodiment, the selenium compound is administered at least 3 days prior to chemotherapy and may be continued during and after the chemotherapy.

### BRIEF DESCRIPTION OF THE FIGURES

FIG. 1 is a representation of the effect of selenium on the antitumor activity of irinotecan (CPT-11) in nude mice bearing HCT-8 colon xenografts. Irinotecan was administered by i.v. push once a week for 4 weeks and methylselenocysteine (MSC) by oral route (p.o.) daily for 28 days with the first dose administered 7 days prior to the administration of irinotecan.

FIG. 2 is a representation of the effect of selenium compounds on the antitumor activity of irinotecan in colon carcinoma and squamous cell carcinoma of the head and neck xenograft tumors for irinotecan alone or in combination with MSC (0.2 mg/mouse/day for 42 days) was administered for 4 weeks. Irinotecan was administered by intravenous (i.v.) push. Irinotecan was started on day 7 after the initiation of the MSC administration. *, ** and *** indicates toxic doses of 50%, 100% and 20% lethality. Animals which survived toxic doses of irinotecan were used to calculate tumor effect.

FIG. 3 is a representation of the effectiveness of MSC and SLC in enhancing the antitumor activity of xenograft bearing A253 and FaDu tumors. Irinotecan was used at a concentration of 100mg/kg.

FIG. 4 is another representation of the effectiveness of two selenium compounds on the antitumor activity of irinotecan. Irinotecan was used at a concentration of 200 mg/kg.

FIG. 5 is a representation of the effect of MSC on the antitumor activity of drugs cisplatin, taxol, cyclophosphamide and doxorubicin on A253 and FaDu tumors for control (●), drug alone (▴) and drug plus MSC (▪).

FIG. 6 is a representation of the effect of selenium on the median tumor weight in rats bearing advanced Ward colorectal carcinoma when treated with control or oxaliplatin alone or in combination with MSC. Oxaliplatin was administered by a single i.v. injection and MSC (0.75 mg/rat/day) by p.o. daily for 21 days with the first dose given 14 days before oxaliplatin treatment. Each group had 8 rats from 2 independent experiments.

FIG. 7 is a representation of the effect of selenium on the antitumor activity of oxaliplatin in rats bearing advanced colorectal carcinoma. Data is presented for oxaliplatin alone and for oxaliplatin in combination with MSC. Oxaliplatin was administered by a single i.v. injection and MSC 0.75 mg/rat/day p.o. daily for 21 days and the first dose started 14 days before oxaliplatin treatment. Each group had 8 rats from 2 independent experiments.

FIG. 8 is a representation of the effect of MSC on the antitumor activity of dxorubicin and oxaliplatin against human A253 and FaDu head and neck xenograft for control, drug alone and for drug in combination with MSC. Each group had 10 mice from 2 independent experiments.

FIG. 9 is another representation of the effect of MSC on the antitumor activity of doxorubicin or oxaliplatin on A253 and FaDu tumors. Doxorubicin was administered by a single i.v. injection and oxaliplatin by i.v. push weekly for 4 weeks. MSC (0.2 mg/mouse/day) was administered by p.o. daily for 14 days with doxorubicin and 28 days with oxaliplatin, and the first dose was started 7 days before chemotherapy. Each group had 10 mice from 2 independent experiments.

FIG. 10 is a representation of the effect of MSC on the antitumor activity of taxotere against human A253 and FaDu head and neck xenografts for control, drug alone and for drug in combination with MSC.

FIG. 11 is a representation of the effect of selenium on mean body weight of nude mice as a function of time when administered alone or in combination with taxotere. Taxotere was administered by a single intravenous injection and MSC was administered at 0.2/mg/mouse/day by p.o. daily for 14 days with the first dose started 7 days before taxotere treatment.

FIG. 12 is a representation of the effect of selenium on taxotere induced toxicity in rats measured as percent of survivors. Taxotere alone or in combination with MSC was administered at the indicated doses. MSC was administered by p.o. daily for 14 days with the first dose started 7 days before taxotere treatment

FIG. 13 is a representation of the effect of pretreatment with selenium on the protection. from irinotecan toxicity.

FIG. 14 is a representation of cure percentages in A253 and FaDu xenograft tumors in response to administration of irinotecan and selenium compound as a function of MSC or SLM concentrations.

FIG. 15 is a representation of total selenium concentrations in nude mice after 7 days of treatment with SLM at various concentrations. The minimum effective dose in attenuating irinotecan toxicity (0.01 mg/day) was associated with a mean selenium concentration of 1200 ng/ml.

FIG. 16 is a representation of the total serum selenium concentration in a patient.

FIG. 17 is representation of the effect of MSC and SLM on augmentation of the cure percentage of irinotecan against FaDu xenografts as a function of time of administration of the selenium compound.

FIGS. 18A and 18B are representations of the antitumor activity of FUra and MSC in nude mice bearing human HCT-8 and HT-29 colon xenografts.

FIGS. 19A and 19B are representations of the effect of FUra or irinotecan alone and in combination with MSC against human HCT-8 (20A) and HT-29 (20B) colon cancer xenografts cure percentages.

FIG. 20 is a representation of the effect of MSC, methylseleninic acid (MSA) and sodium selenite (SS) on the cure percentages of irinotecan in A253 and FaDu head and neck tumor xenografts.

### DETAILED DESCRIPTION OF THE INVENTION

The term “therapeutic dose” as used herein means the dosage of a therapeutic agent that is acceptable for use clinically with respect to its toxicity without the co-administration of selenium compounds.

The term “cure” as used herein means the complete disappearance of a tumor. A tumor is considered to have completely disappeared when it is undetectable by palpation.

Anti-cancer agents generally fall into one or more of the following functional categories: antihormones, antifolates, antimicrotubule agents, alkylating agents, antimetabolites, antibiotics, topoisomerase inhibitors and antivirals. In the present invention it was observed that while augmentation of tumor shrinkage was observed for several chemotherapeutic agents, the effect was not durable for all chemotherapeutic agents. Tumor cures were observed only with specific agents even within the same class of agents. For example, MSC and SLM were observed to enhance the cure rates of xenografis bearing tumors treated with irinotecan, but not with 5-FU. Although, selenium in combination with Fura produced some effect, this effect was not long-lasting and did not result in cures, a clinically important endpoint.

Thus the cure augmentation effect the combination of selenium on the anti-tumor agent is surprisingly drug specific. Topotecan, Camptothecin and irinotecan are all Topoisomerase I inhibitors. However, only the cure effect of irinotecan was enhanced by selenium. Similarly, taxol, taxotere are inhibitors are microtubule inhibitors, but the augmentation of the cure effect was achieved only with taxotere and not with taxol.

In the present invention, it was also observed that administration of selenium compounds, MSC and SLM, augments the antitumor activity of anticancer agents. Data is presented for in vivo studies in xenograft bearing animals. Additionally, clinical studies have been carried out which further confirm augmentation of antitumor activity of anticancer agents by administration of selenium. In one study, patients were treated with a combination of a high dose of seleno-L-methionine (SLM) (2200 μg) orally daily starting I week prior to starting chemotherapy (irinotecan once a week). One patient whose tumor was previously progressing while receiving irinotecan alone responded with more then 50% shrinkage in the tumor when SLM was added and high serum selenium levels were reached. The protection against toxicity is supported further by the fact that this patient had complete reversal of hair loss once the selenium levels reached a level higher then the threshold identified in the mice studies. The patient has not experienced any significant bone marrow or intestinal toxicity despite being on ongoing full dose chemotherapy for 1 year.

We have also noted other cases of unexpected disease stabilization and minor shrinkage with the addition of SLM to irinotecan. These include: a case of refractory colon cancer with >25% disease shrinkage allowing resection and currently free of disease (6 months plus), a case of pancreatic cancer refractory to standard treatment with an ongoing disease stabilization of than 4 months, a case of refractory stomach cancer with ongoing disease stabilization of more than 8 months, and a case of lung cancer with disease stabilization of 6 months duration. These favorable outcomes are highly unusual in these advanced chemotherapy-resistant tumors and are likely secondary to the contribution of SLM.

The method of the present invention comprises administering to an individual, in need of such a treatment, one or more anticancer agents and one or more selenium compounds selected from the group consisting of MSC and SLM. The selenium compound is preferably administered prior to administration of the anticancer agent. In one embodiment, the selenium compound is administered at least 3 days and more preferably about one week prior to administration of the anticancer agent. By combining chemotherapy with the administration of selenium compounds, the antitumor toxicity of the chemotherapeutic agent can be increased resulting in higher cures.

The present invention also provides a method for inhibiting the growth of a tumor that has proven to be refractory to anticancer agents comprising administration of a selenium compound and the anticancer agent. The selenium compound is preferably administered prior to the start of the administration of the anticancer agent.

It is preferable to use selenium from organic forms since these are known to be less toxic. Examples of useful selenium compounds from organic forms include methylselenocysteine (MSC) and seleno-L-methionine (SLM). The doses of selenium compounds are in the range of about 200 μg/person to about 3.6 mg/person and maybe administered daily for 1 year or longer.

In one embodiment, the selenium dose is such as to maintain serum selenium concentrations of at least about 1200 ng/ml. In one embodiment, a dose of at least 2000 μg/day of selenium can be used as a chemotherapy-potentiating agent. In another embodiment, a dose of at least 2200 μg/day can be used. In another embodiment, a loading dose of 3200 to 5600 μg p.o. BID×1 week prior to initiation of irinotecan can achieve selenium concentrations >1200ng/ml. A maintenance dose in the range of 2800 to 5600 μg/day starting day 8 to can be used to maintain a serum selenium concentration >1200 ng.

The present invention comprises the steps of combining chemotherapy with the administration of selenium. The dosage and administrative regimens of irinotecan and/or taxotere are well within the purview of those skilled in the art. Selenium administration is preferably initiated before the start of chemotherapy and can be continued during the chemotherapy and after cessation of chemotherapy.

To demonstrate the effect of selenium in reducing the toxic effect of chemotherapeutic agents, studies were carried out in tumor bearing nude mice. It should be noted that while previous studies have reported an effect of selenium on reducing toxicity (cardiotoxicity) of some anticancer agents in vitro, such studies do not permit an assessment of the effect of selenium on the efficacy of anticancer agents.

In one embodiment of the present invention, it was determined that methylselenocysteine (MSC) and seleno-L-methionine (SLM) are effective agents in augmenting the antitumor activity of anticancer agents. Agents representing five different classes of clinically approved compounds were tested. Thus, the chemotherapeutic agents tested were irinotecan, topotecan, camptothecin (topoisomerase I inhibitor); doxorubicin, (topoisomerase II inhibitor), FU (DNA synthetic inhibitor); taxol and taxotere (microtubule inhibitor) and cisplatin and oxaliplatin (DNA alkylating agents). The two selenium containing compounds were evaluated in xenograft tumors in mice for the various chemotherapeutic agents. The in vivo effects were observed using non-toxic doses of the selenium containing compounds (about 0.2 mg/mouse/day or lower).

It should also be noted that selenium containing compounds, 5-methylselenocysteine (MSC) and seleno-L-methionine (SLM) were found not to be toxic when 0.2 mg/mouse/day for 28 days was administered orally to normal nude mice and are effective modulators of toxicity induced by anticancer drugs. In one embodiment, it is demonstrated that Selenium containing compounds, MSC and SLM potentiate the cure rate of irinotecan in xenografts bearing drug sensitive and relatively resistant tumors. Further, MSC potentiates the antitumor activity of taxol, cisplatin (CDDP), oxaliplatin, cyclophosphamide, taxotere and doxorubicin (Dox) of xenografts bearing human A253 and FaDu squamous cell carcinoma of the head and neck tumors. However, it was observed that when cure rates were evaluated, selenium increased the efficacy of only irinotecan and taxotere. While not intending to be bound by any particular theory, it is considered that potentiation of the efficacy of anticancer drugs is associated with increased antitumor activity and decreased toxicity.

The present invention can be used for treatment of tumors including, but not limited to, adenocarcinomas, melanomas, lymphomas, sarcomas, leukemias, and different organ tumors like lung, breast, ovarian, head and/or neck, prostate, cervical, endometrial, colorectal, gastric, liver, fallopian tubes, esophagus, small intestine, pancreas, kidney, adrenal, vaginal, vulvar, brain and testicular tumors. The combination regimen of an antitumor agent and selenium may be used with other anticancer therapies such as radiation, surgery and immunotherapy. This invention can be used for achieving antitumor effect in mammals including humans, mice, rats, dogs etc.

The present method is an improvement over existing methods. In the present invention, reduction of toxicity was observed for bone marrow, gastrointestinal, hair, kidneys and bladder. Although cytotoxic chemotherapy may be associated with improved survival in a variety of cancers, this treatment modality is associated with significant toxicities that impact patients' quality of life and interfere with dose intensity. The addition of an agent that reduces bone marrow toxicity, alopecia, and diarrhea (and possibly other toxicities) will have a major positive impact on patients' tolerance to chemotherapy and will improve their quality of life. No other agent is proven at this time to reduce these combined toxicities.

Chemotherapy is associated with the eventual development of resistance and ultimate disease progression in the majority of advanced malignancies. We have shown that SLM overcomes chemotherapy resistance in animal models. We have also shown reversal of irinotecan toxicity in a patient with advanced colon cancer who achieved target selenium (>1200 ng/ml) concentrations with SLM. Accordingly, selenium can potentiate the effects of chemotherapy in patients with cancer. These benefits may extend from increasing cure rates in chemosensitive tumors such as testicular cancer and patients receiving adjuvant therapy to prolongation of overall survival in patients with other advanced resistant malignancies.

The following examples are provided below to illustrate the present invention. These examples are intended to be illustrative and are not to be construed as limiting in any way.

### EXAMPLE 1

This example describes the administration schedules for the selenium compounds and the anticancer agents used in Examples 2-7. In addition, the tumor xenografts established are also described.

5-Methylselenocysteine (MSC). Two schedules were evaluated: 1) in combination with irinotecan, MSC (0.2 mg/mouse/d×28) was administered orally for 28 days with the first dose administered daily for seven days prior to the weekly drug administration and 2) in combination with other drugs, MSC was administered orally (0.2 mg/mouse/d×14) daily for 14 days with the first dose administered daily for seven days prior to the single i.v. administration of taxol, CDDP, Dox, taxotere and cyclophosphamide.

Anticancer drug administration. The anticancer drugs administration schedule was as follows.
i. Irinotecan (CPT-11), weekly i.v. push for four (4) weeks,
ii. Taxol, single i.v. push,
iii. Cisplatin (CDDP), single i.v. push.
iv. Doxorubin (Dox), single i.v. push.
v. Cyclophosphamide, single i.v. push.
vi. Oxaliplatin, single i.v. push
vii. Taxotere, single i.v. push

Tumor Xenografts. The tumor xenografts (all tumors have a doubling time of approximately 3 days) were initially established by implanting subcutaneously 10⁶ cultured cells and passed several generations by transplanting 50 mg or more non-necrotic tumor tissues before treatment. The following tumor xenografts were established.
i. HCT-8: poorly-differentiated colon carcinoma, expressing wild type p53
ii. HT-29: well-differentiated colon carcinoma expressing mutant p53
iii. A253: well-differentiated squamous cell carcinoma of the head and neck (SCCHN) expressing no p53
iv. FaDu: poorly-differentiated squamous cell carcinoma of the head and neck (SCCHN) expressing mutant p53

### EXAMPLE 2

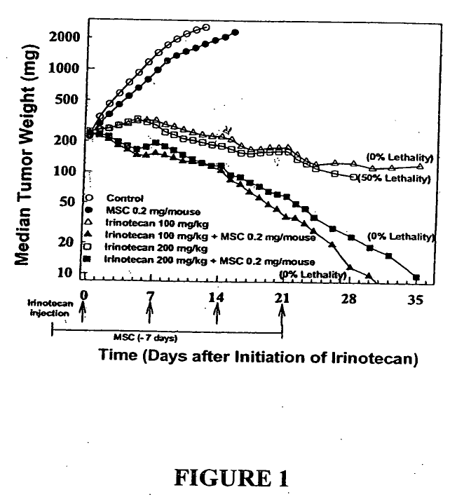

This example describes the evaluation of the effect of selenium containing compounds on the antitumor activity of irinotecan. In this example, the effect of selenium on the antitumor activity of irinotecan was determined. Irinotecan was administered at 100 mg/kg/wk for 4 weeks (MTD) and 200 mg/kg/wk for 4 weeks (toxic) alone and in combination with 0.2 mg/mouse/d of MSC for 28 days to nude mice bearing HCT-8 colon xenografts. The results are shown in FIG. 1. The data indicate that although the kinetics of response to 100 mg/kg and 200 mg/kg of irinotecan in combination with MSC is similar with complete tumor regression achieved within one to two weeks after termination of treatment, MSC offered complete protection against lethal doses of irinotecan (200 mg/kg). All the animals survived treatment with irinotecan in combination with MSC compared with 50% survival of animals treated with irinotecan alone. Thus, MSC potentiates the efficacy of irinotecan by increasing cure rate and by decreasing toxicity.

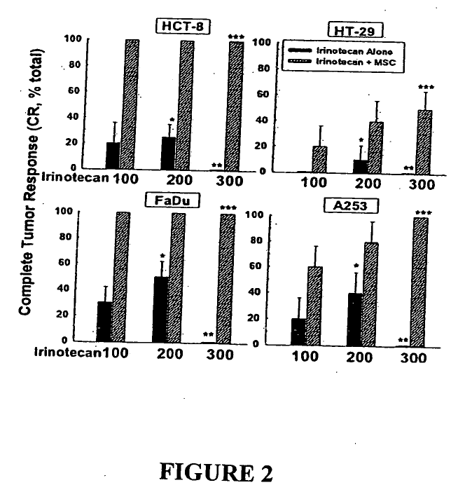

The data in FIG. 2 is the summary of cures of xenografts treated with different doses of irinotecan ±MSC in two colon carcinomas (HCT-8 and HT-29) and squamous cell carcinoma of the head and neck (FaDu and A253) xenograft tumors. The maximum tolerated weekly dose of irinotecan is 100 mg/kg/wk for 4 weeks. The 200 mg/kg and 300 mg/kg are lethal doses where 50% and 100% of animals did not survive the four weeks of therapy, respectively. With the 100 mg/kg/wk for 4 weeks irinotecan (MTD), the cure rate was increased from 20% to 100% in HCT-8, from 0% to 20% in HT-29, from 30% to 100% in FaDu and from 20% to 60% in A253 xenografts. The data in FIG. 2 also indicate that while HT-29 (colon) and A253 (SCCHN) tumors are less sensitive to the MTD of irinotecan, than HCT-8 and FaDu tumors, administration of higher doses of irinotecan yield higher cure rates with 200 and 300 mg/kg/wk for 4 weeks to 40% and 50%, respectively in HT-29 and to 80% and 100% in A253 tumors, respectively. While the increase in cures by MSC with 200 mg/kg irinotecan was achieved without toxicity (lethality), increased cure by MSC with 300 mg/kg irinotecan was associated with 20% lethality. In contrast, 200 and 300 mg/kg irinotecan was associated with 50% and 100% lethality. The data in FIG. 2 demonstrates further that MSC effectively modulates the cure rate of irinotecan in several human xenograft tumors with differential response to the MTD of irinotecan.

### EXAMPLE 3

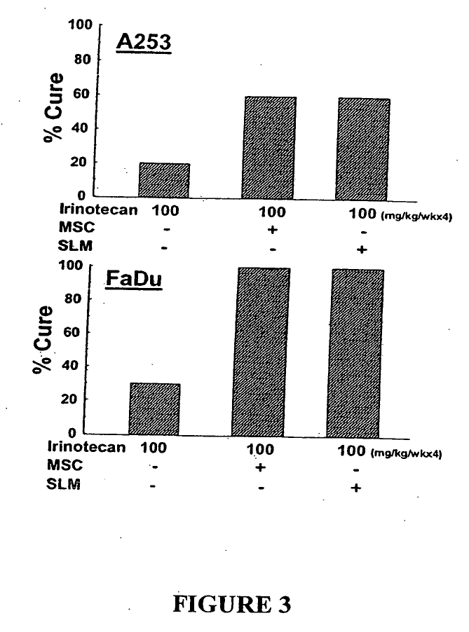

This example describes the comparative antitumor activity of MSC and SLM in combination with the maximum tolerated dose of irinotecan. This example demonstrates that any selenium compound can be used to enhance the effects of the antitumor agents. As an illustration, MSC and SLM were used in combination with irinotecan. The results, shown in FIG. 3, represent a comparative evaluation of the effect of MSC and SLM (0.2 mg/mouse/d×28) on the antitumor activity of irinotecan (100 mg/kg/wk×4). In both A253 and FaDu, MSC and SLM produced similar potentiation of the antitumor activity of irinotecan, indicating that the effect is not specific for MSC.

### EXAMPLE 4

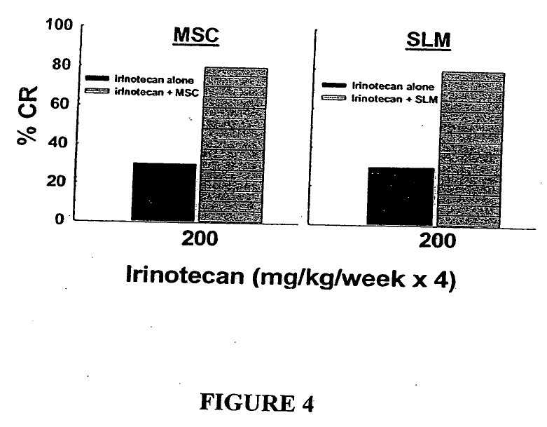

This example describes the comparative evaluation of MSC and SLM as selective modulator of the antitumor activity and toxicity of irinotecan administered at twice the maximum tolerated dose. This example demonstrates that since the selenium compounds reduce the toxicity of the antitumor agents, the dose of the antitumor agents that can be administered can be increased. To determine whether therapeutic synergy achieved with irinotecan in xenografts is specific for MSC, the antitumor activity of irinotecan with and without two selenium-containing compounds, MSC and SLM were compared in xenografts bearing A253 (SCCHN) tumors for the maximum tolerated dose (200 mg/kg/wk×4). The results are presented in FIG. 4. The data compare the antitumor activity of MSC with SLM in combination with irinotecan (200 mg/kg/wk×4). The results outlined in FIG. 4 indicate that MSC and SLM produced equal potentiation of the antitumor activity of irinotecan with 80% of the treated animals were cured of their disease with no toxicity, significant lethality was observed in approximately 50% of the animals treated with this dose of irinotecan. Of the 50% of animals who survived treatment with irinotecan (200 mg/kg/wk×4) in combination with MSC or SLM 80% were cured compared with 40% cures of animals treated with irinotecan alone. Thus, MSC and SLM are equally effective in selective modulation of antitumor activity of irinotecan.

A summary of the effect of MSC in potentiation of the antitumor activity of irinotecan is presented in Table 1.

TABLE 1
5-Methylselenocysteine (MSC, 0.2 mg/mouse/d × 28) increase the cure rate (CR)
without toxicity of xenografts treated with Irinotecan

| | MSC | | | | | |
|---|---|---|---|---|---|---|
| Irinotecan | (.02 mg/ | Survivor | % CR of Surviving Animals | | | |
| mg/kg/wk × 4 | mouse/d × 28) | (%) | HCT-8 | HT-29 | A253 | FaDu |
| 100 | − | 100 | 20 | 0 | 20 | 30 |
| 100 | + | 100 | 100 | 20 | 60 | 100 |
| 200 | − | 50 | 30 | 10 | 40 | 50 |
| 200 | + | 100 | 100 | 10 | 80 | 100 |
| 300 | − | 0 | †NE | NE | NE | NE |
| | | | (lethal) | (lethal) | (lethal) | (lethal) |
| 300 | + | 80 | 100 | 50 | 100 | 100 |

†NE, were not evaluable since 100% of the animals did not survive the 4 weeks of treatment.

The data shows the antitumor activity (cures) of irinotecan alone and in combination with MSC against xenografts bearing human tumors. In all four tumors, MSC potentiates significantly the antitumor activity of irinotecan. Since it was not possible to assess accurately tumor response to toxic dose of irinotecan (200 and 300 mg/kg/wk×4) due to lethality, demonstrated ability of MSC to protect normal tissues against toxic doses of irinotecan provided the opportunity for delivering the higher doses of irinotecan resulting in increased cure rates in the four human tumor xenografts evaluated. While the 300 mg/kg/wk×4 administration of irinotecan resulted in 100% lethality, in combination with MSC, % lethality was reduced to 20%.

### EXAMPLE 5

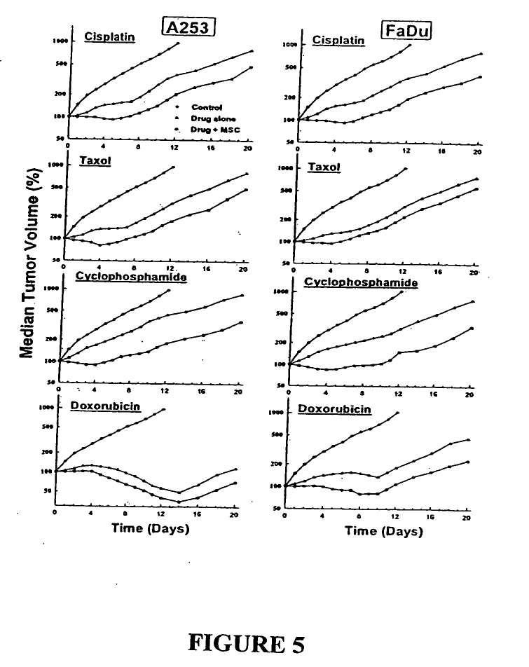

This example describes the modulation of the antitumor activity of anticancer drugs by MSC in mice bearing human tumors. This embodiment demonstrates that selenium compounds can be used to potentiate the antitumor activity of a broad spectrum of antitumor agents. To determine whether modulation of the therapeutic efficacy and cure of irinotecan by MSC is drug specific, the antitumor activity of drugs, representing different classes and mechanisms of action were evaluated alone and in combination with non-toxic doses and schedules of MSC (0.2 mg/mouse/d×14) in xenografts bearing human A253 and FaDu (SCCHN) tumors (FIG. 5). The data in FIG. 5 represent the use of the MTD doses of cisplatin (8 mg/kg×1), cyclophosphamide (100 mg/kg×1), taxol (35 mg/kg ×1.) and doxorubicin (10 mg/kg×1). The results indicate that MSC potentiates the antitumor activity of each drug in xenografts bearing A253 and FaDu tumors. Potentiation of the antitumor activity by MSC was not associated with any increased toxicity with these clinically important chemotherapeutic agents. The data in FIG. 5 clearly demonstrates that MSC modulation of the antitumor activity of anticancer drugs covers a broad spectrum of anticancer agents.

### EXAMPLE 6

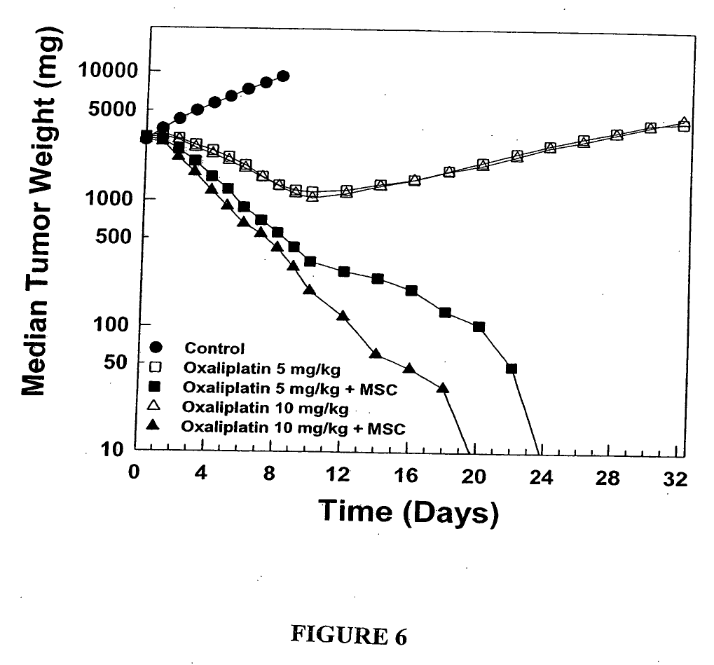

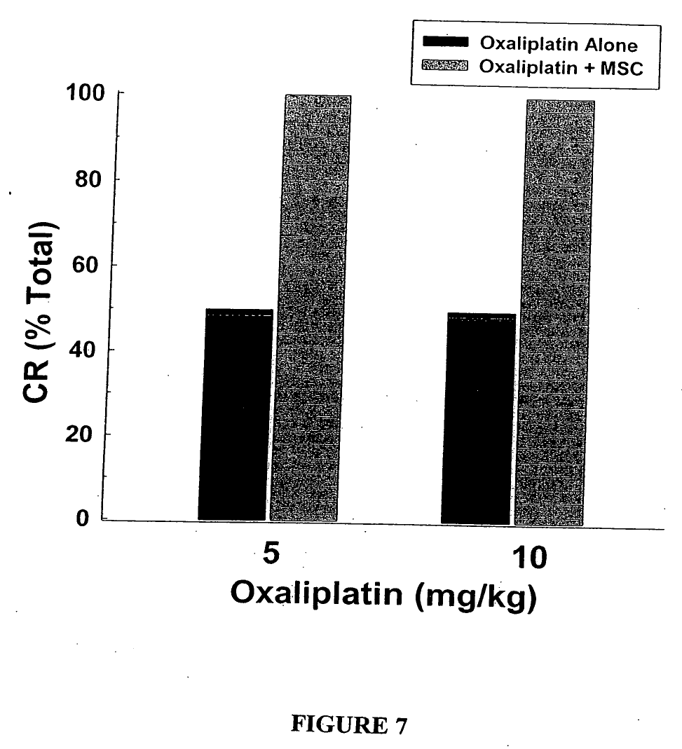

This example describes the effect of Selenium on the antitumor activity of oxaliplatin in rats bearing advanced ward cancer and in nude mice bearing human squamous cell carcinoma for the head and neck A253 and FaDu xenografts. This example further demonstrates that selenium enhances the antitumor activity of a wide spectrum of antitumor agents. In this example, the effect of selenium on the antitumor activity of oxaliplatin and doxorubicin was tested as follows. The effect of selenium was determined on the antitumor activity of oxaliplatin in rats bearing advanced ward colorectal carcinoma (3000 mg). Rats received MSC (0.75 mg/rat/d) or saline fourteen days prior to single i.v. injection of oxaliplatin of 5 and 10 mg/kg with continuous daily oral administration of MSC for additional seven days for a total of 21 days of MSC and oxaliplatin is administered on day 14 after saline and MSC treatment. The data indicate that while oxaliplatin at 5 and 10 mg/kg exhibited similar antitumor activity (tumor growth inhibition), rats treated with the combination of oxaliplatin and MSC, however, demonstrated significant enhancement of tumor growth inhibition since all of the animals were without detectable tumor (cures) on about days 20-24 (FIG. 6). Of interest, optimal cure rate was only detected at approximately three weeks after the i.v. administration of a single dose of oxaliplatin (delayed antitumor effect). Further, lack of dose response with oxaliplatin is clearly evident since 5 (MTD) and 10 mg/kg yielded similar cure rate (FIG. 7). In addition, while 10 mg/kg Oxaliplatin was toxic, the observed high cure rate of oxaliplatin with MSC was without any detectable toxicity (weight loss and diarrhea). Thus, MSC is highly selective and in therapeutic trials synergistic when combined with oxaliplatin in this rat ward colorectal tumor.

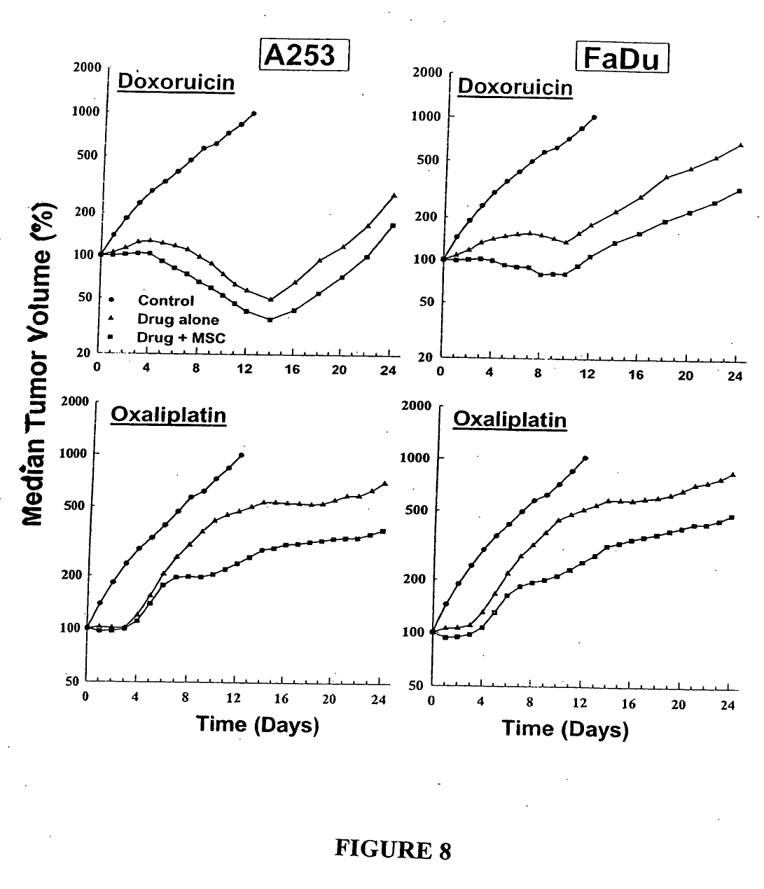

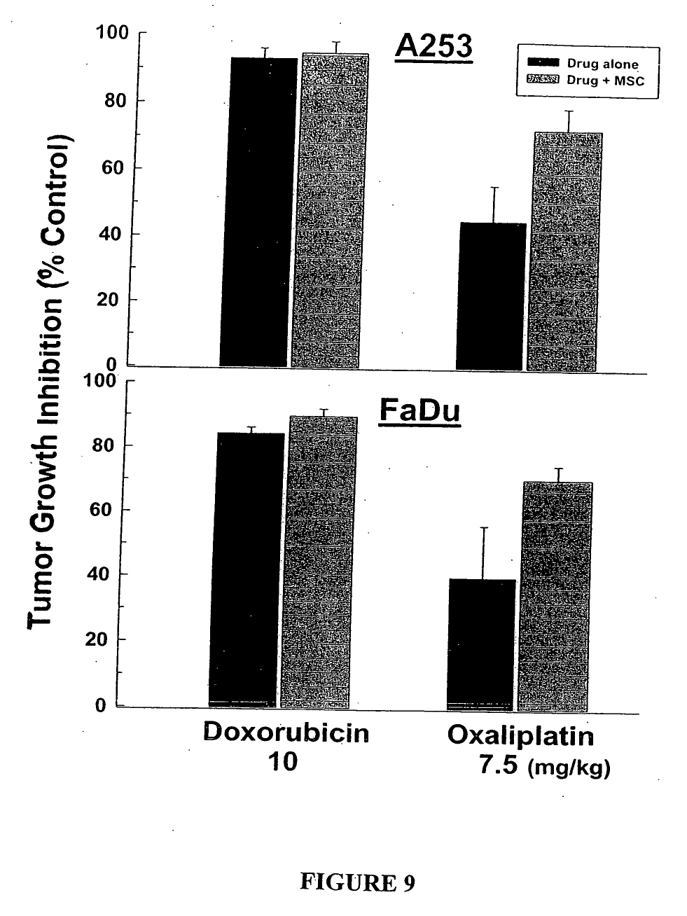

In another illustration of this embodiment, the effect of selenium on the antitumor activity of oxaliplatin and doxorubicin was tested on human squamous cell carcinoma xenografts. The data in FIG. 8 is a graphic representation of the kinetics of the antitumor response of xenograft tumors (A253/FaDu) treated with doxorubicin (10 mg/kg×1) and oxaliplatin (15 mg/kg×1) alone and in combination with MSC. In mice, MSC was orally administered at 0.2 mg/mouse/d with the first daily dose administered seven days prior to single i.v. administration of drug and continuous for additional seven days for a total MSC treatment of 14 days. The results indicate that MSC potentiates the antitumor activity of both drugs in both A253 and FaDu xenografts (FIGS. 8 & 9). The observed increase with MSC in the antitumor activity of oxaliplatin and doxorubicin was not associated with any toxicity. Thus, MSC is highly selective in potentiation of the antitumor activity of oxaliplatin and doxorubicin in xenografts bearing A253 and FaDu tumors.

In summary, the maximum tolerated doses of oxaliplatin and doxorubicin alone and in combination with MSC were compared and the data indicate that the MTD of drugs is higher when combined with MSC due to MSC protection of normal tissues for drug induced toxicity. These results are summarized in Table 2.

TABLE 2

MTD
(mg/kg)

| Drug | MSC | Rats | Mice |
|---|---|---|---|
| Doxorubicin | − | 6 | 10 |
| Doxorubicin | + | 9 | 12.5 |
| Oxaliplatin | − | 15 | 7.5 |
| Oxaliplatin | + | 20 | 12.5 |
| Taxol | − | 50 | 35 |
| Taxol | + | 100 | 75 |

### EXAMPLE 7

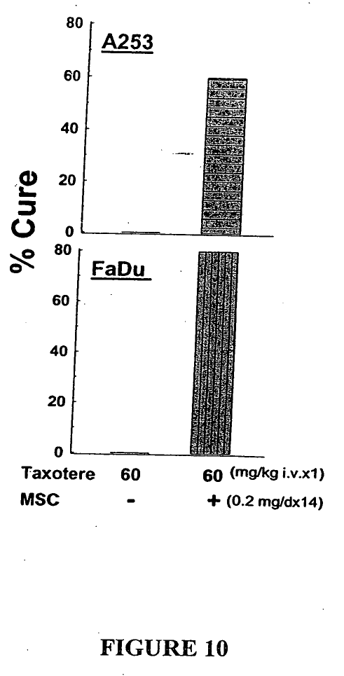

This embodiment demonstrates that selenium can augment the antitumor activity of another anticancer agent, i.e., taxotere. To illustrate this embodiment, the effect of taxotere was evaluated alone and in combination with MSC (0.2 mg/mouse/d×14) in xenografts bearing human A253 and FaDu (SCCHN) tumors. Toxotere was administered by a single i.v. injection and MSC by p.o. daily for 14 days with the first dose started 7 days before taxotere treatment. The results (FIG. 10) show that while both tumors were insensitive to the MTD dose of taxotere (60 mg/kg), the combination of MSC and taxotere increased the number of animals cured of their tumors to 60% in A253 xenografts and to 80% in FaDu bearing xenografts. These results indicate that MSC potentiates the antitumor activity of taxotere and is capable of reversal of resistance of these tumors to taxotere.

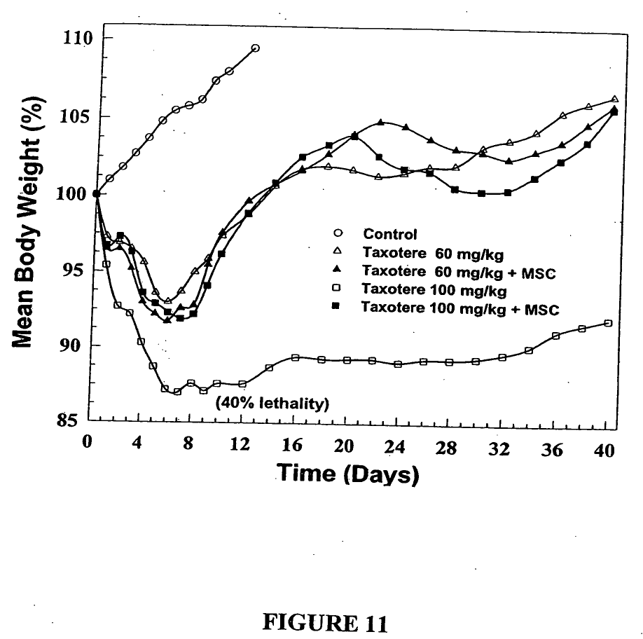

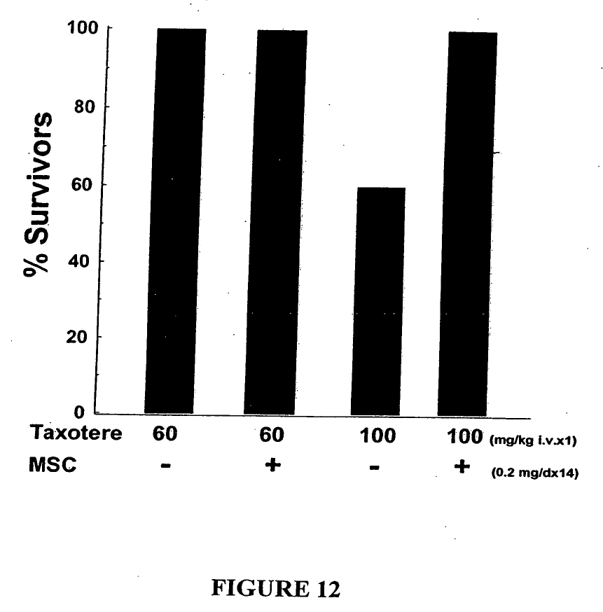

Furthermore, it was also observed that selenium protects against taxotere induced toxicity. Taxotere was administered to nude mice at non-toxic (60 mg/kg) or toxic (100 mg/kg) dose by a single i.v. injection and MSC was administered by oral route daily for 14 days with the first dose of MSC started before taxotere treatment. The results on mean body weight are shown in FIG. 11. While 100 mg/kg taxotere resulted in approximately 15% loss of total body. weight, in combination with MSC, the weight loss was insignificant and similar to untreated animals. The effect on survival is shown in FIG. 12. Again, while.100 mg/kg taxotere resulted in 40% lethality, in combination with MSC, 100% of the animals treated with 100 mg/kg taxotere survived with no signs of toxicity. (FIG. 12).

### EXAMPLE 8

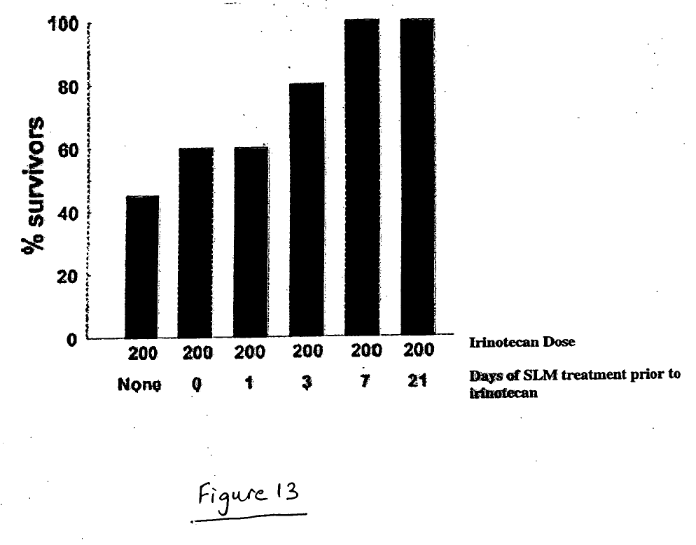

This example demonstrates that pre-treatment with SLM or MSC enhances the potentiation effect of selenium. Nude mice (5 mice/group; experiment repeated ≧3 times) were treated with 0.2 mg/mouse/day of SLM (data replicated with MSC) for 21, 7, 3, 1, or 0 days prior to initiation of weekly IV irinotecan at 2 ×the MTD (at 200mg/kg/week×4). 100% survivorship was noted for pretreatment with SLM for both the 7 and 21 days pretreatment groups. 3 days pre-treatment was associated with 80% survivorship while 0-1 days were associated with 60% (45% survivorship for irinotecan alone) (FIG. 13). Thus, the optimal duration of pre-treatment with SLM 0.2mg/mouse/day for maximum protection against irinotecan lethality was identified to be at least 1 week.

### EXAMPLE 9

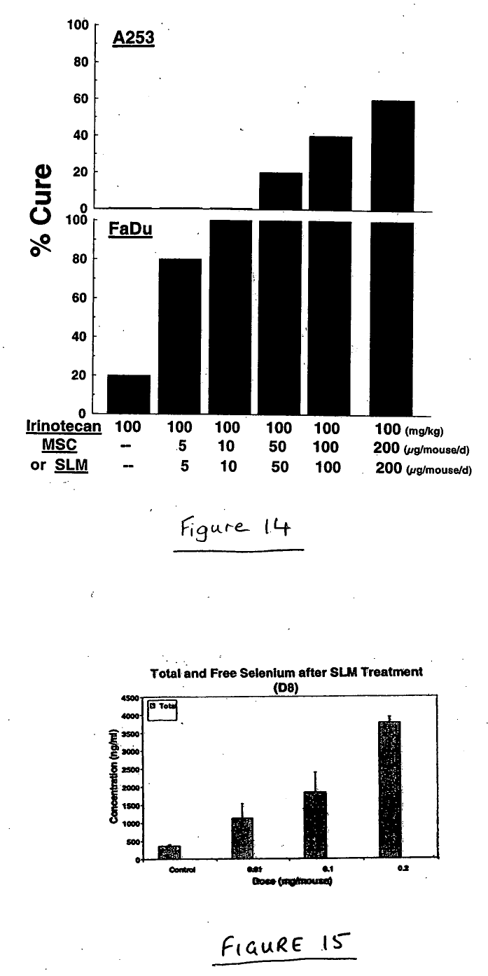

Serum concentrations of selenium after 1-week treatment with SLM at different doses were evaluated in order to identify the lowest biologically effective serum selenium concentration to provide optimal protection against irinotecan toxicity. Sera were collected from mice treated for 7 days with SLM at 0.01, 0.1, and 0.2mg/day. Selenium concentrations were tested at Roswell Park Cancer Institute (RPCI). The mean concentration achieved on day 8 of the minimum effective dose of SLM (0.01 mg/day) was 1200 ng/ml (FIG. 14). Thus, it would be desirable to achieve a serum concentration 1200 ng/ml of selenium prior to administration of chemotherapy to be able to test adequately the potential of toxicity attenuation of SLM (or MSC) in humans.

### EXAMPLE 10

This example describes that higher selenium concentrations have greater potentiation of antitumor activity when combined with irinotecan in de novo resistant xenograft tumors in mice. After establishing 0.01 mg/day (1200 ng/ml serum selenium concentration) of SLM as the minimum necessary dose to achieve protection against irinotecan toxicity, we tested if further dose-escalation has any favorable antitumor activity when combined with irinotecan. Two tumors were tested (xenografts in nude mice): A253 (resistant head and neck tumor) and FaDu (sensitive head and neck tumor). All mice were treated with 100 mg/kg of irinotecan starting 1 week after SLM or MSC initiation and given weekly ×4 (5 mice/treatment arm; experiment. repeated 3 times). While the lowest effective dose of SLM or MSC in attenuating toxicity (0.01 mg/day or 10 μg/day) increased the cure rates of FaDu xenografts to 100%, this dose did not result in any notable increase in cure rates with the more resistant cell line A253. However, the administration of SLM or MSC at a dose of 0.1 mg/day (100 μg/day; equivalent serum concentrations of selenium of 1800 ng/ml) or higher resulted in a synergistic antitumor activity in A253 manifesting as an increase in cure rates (FIGS. 14 and 15).

### EXAMPLE 11

This example describes clinical studies demonstrating the potentiation of the effect of chemotherapeutic agents by selenium. Patients whose tumors had proven to be refractory to anticancer agents were used in these studies. A phase one study of irinotecan in combination with high dose SLM was conduced. Patient eligibility included age ≧18 years, performance status ECOG 0-1, normal organs function (including liver, kidneys, and bone marrow), and the documentation of a refractory solid tumor. SLM was started 1 week prior to initiation of irinotecan. Irinotecan was administered i.v. over 90 minutes once weekly ×4 and repeated every 6 weeks. The dose of SLM was fixed at 2200 μg/day at all dose-levels. Irinotecan was escalated by 30% increments starting at the standard dose of 125 mg/m²/week ×4. The second dose-level (DL) of irinotecan was 160 mg/m²/week. A standard 3-3-dose escalation was followed and intra-patient dose-escalation was not allowed. Dose escalation was to stop once ≧2 patients at a dose level developed dose-limiting toxicities and this dose level was to be labeled as an intolerable dose. The maximum tolerated dose was defined as the dose level below the intolerable dose at which ≧1 patient out of 6 developed a dose limiting toxicity (DLT). DLT was defined as any grade 3 or 4 non-hematological toxicity (grade 3 diarrhea had to last 24 hours), any febrile neutropenia, grade 4 thrombocytopenia, grade 4 neutropenia for more than 1 week, dose delay of more than 2 weeks because of toxicity, or omission of more than 1 dose of irinotecan because of toxicity. The study was also designed to evaluate the pharmacokinetics of selenium and irinotecan.

10 evaluable patients were treated in this study (Male/Female: 6/4, median age: 59; ECOG 0/1: 3/7; prior chemo: 10). At dose-level 2 (irinotecan 160mg/m²/week ×4 every 6 weeks), 3 out of4 patients had G3 diarrhea (DLT). This dose was deemed non-tolerable. This dose level is 30% higher than the standard dose of 125 mg/m².

At dose-level 1 of irinotecan (FDA recommended dose and schedule) minimum toxicity was noted. Only 1 out of 6 patients had a grade 2 diarrhea and no grade 3 or above diarrhea were noted. Only 1 out 6 patients at dose level 1 had ≧grade 3 neutropenia. No dose limiting toxicities were seen on dose level 1.

Responses were evaluable in 8 patients: 1 patient with prior irinotecan-refractory colon cancer had a partial response that is ongoing (1 year now). This patient had previously failed 3 lines of chemotherapy for colon cancer (irinotecan, 5-FU, oxaliplatin, FUDR). This patient has a documented partial response that is ongoing and is associated with a marked reduction in CEA levels. It noteworthy that this patient has had the highest selenium levels among all other patients (likely because of relatively light weight of 40kg). The patient's selenium concentrations were the highest among all patients and reached a level 1200ng/ml by 3 weeks from initiation of SLM (FIG. 16). While not intending to be bound by any particular theory, it is considered that high serum selenium levels are necessary to result in toxicity prevention. This is reflected by the fact that the patient's maximum toxicity on cycle 1 was grade 2 neutropenia and diarrhea despite being on dose-level 2 (the only patient on dose-level 2 who did not have a grade 3 toxicity). On cycle 2 and beyond, the patient received the standard dose of irinotecan and did not develop any grade 2 or higher toxicity.

Four other patients also demonstrated stabilization of disease after a combination of selenium and had a stable disease. One patient with gastric cancer who had failed prior cisplatin, taxotere, and capecitabine has ongoing disease stabilization (>6 months). One patient with NSCLC with prior progression on carboplatin and paclitaxel had disease stabilization for 6 months. One patient with pancreatic cancer, resistant to gemcitabine, has ongoing disease stabilization (4 months). One patient with colon cancer had a minimal response followed by surgery and is currently free of disease.

In 9 patients evaluated to date, selenium absorption was variable and trough levels on day 8 ranged from 363 to 985 ng/ml (median, 544). The day 8 PK data indicate a tₘₐₓ between 2 and 8 h (median 3 h) and Cₘₐₓ between 457 and 1107 ng/ml (median 726). The mean (SD) serum half-life was 183 (94) h, and CLt/F 0.10 (0.04) L/h. Modeling of data suggests steady state attainment after ˜30 days in the average patient, with a median steady state level of 844 (range 585 -1300) ng/ml. The plasma levels of selenium attained by day 8 when irinotecan treatment starts are well below the <1200 ng/ml level shown to be protective in animal model, which may account for the inability to dose escalate irinotecan in this trial. One patient (the patient with an ongoing partial response) attained target concentrations of selenium (FIG. 16) after 3 weeks of treatment. She has been on study for almost 1 year now and her serum selenium concentration have stabilized between 2250 and 2750 ng/ml (plateau reached).

Our data support the use of SLM as a chemotherapy antitumor activity potentiating agent in cancer (across different tumor types). These data also support the use of dose >2200 mcg of SLM per day to achieve adequate concentrations in all the treated patient population. A two-compartment pharmacokinetic model was fit to the selenium concentration data of each subject on our study over the entire study period (WinNonlin Pro, version 4.1). This two-compartment model was determined to be superior to other models evaluated, based on Akaike's Information Criterion, and fit the experimental data well. The pharmacokinetic parameters determined for each individual subject (from the true data) were then used to simulate predicted selenium concentrations over several months of dosing. Various candidate loading and maintenance dosage regimens were tested for their ability to achieve the selenium target, while avoiding excessive concentrations. Each candidate regimen was compared across all patients to identify the dosage regimen(s) predicted to best achieve the desired targets. Various candidate loading and maintenance dosing regimens were simulated in each individual patient. Due to the long half-life, accumulation of selenium in the body is slow, taking 1 to 2 months to achieve steady-state concentrations. Therefore, a loading dose was considered to achieve ‘target’ serum selenium concentrations within 7 days, prior to the initiation of irinotecan administration. Through this analysis we have determined that a loading dose of 3200 to 5600 μg p.o. BID ×1 week prior to initiation of irinotecan can achieve selenium concentrations >1200 ng/ml. The required maintenance dose is estimated in the range of 2800 to 5600 μg/day starting day 8 to maintain a serum selenium concentration >1200 ng. It is likely that the upper range of the doses estimates (5600 μg p.o. BID ×1 week followed by a maintenance of 5600 μg p.o. daily) will result in selenium concentration >1800 ng/ml-serum concentrations associated with optimal antitumor potentiation in resistant tumors in vivo.

### EXAMPLE 12

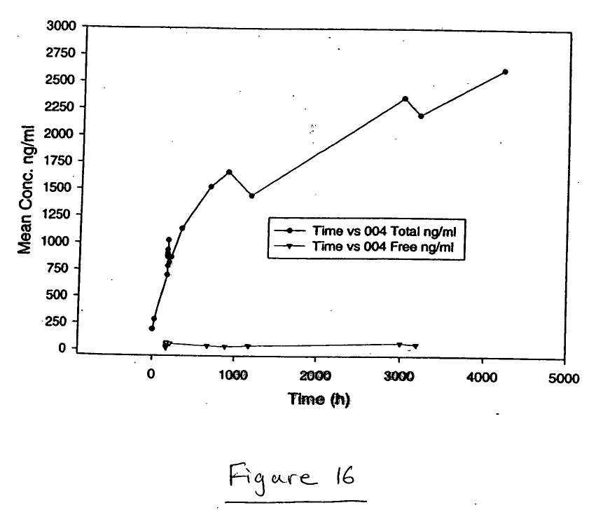

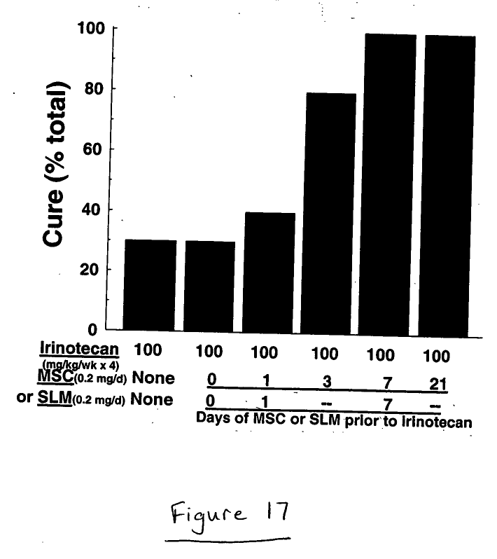

This example demonstrates that optimal cures are obtained when the selenium compound is administered at least 3 days prior to the administration of the anti-tumor agent. MSC or SLM was administered daily starting at 0 days, 3 days, 7 days or 21 days prior to administration of irinotecan. As shown in FIG. 17, simultaneous administration of selenium or administration 1 day prior to irinotecan was not effective. However, when selenium was administered starting at 3 days prior to irinotecan, a significant effect on percent of cures was observed. Optimal effect was seen when selenium administered was started 7 or 21 days prior to irinotecan.

### EXAMPLE 13

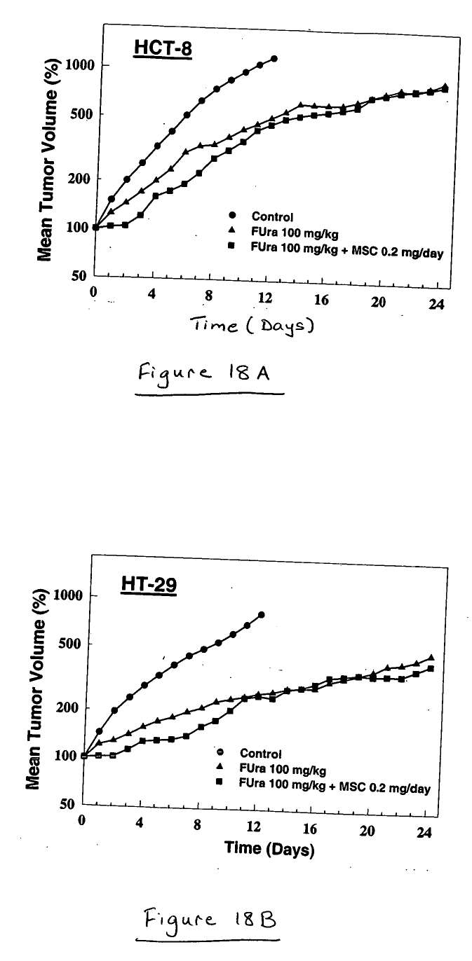

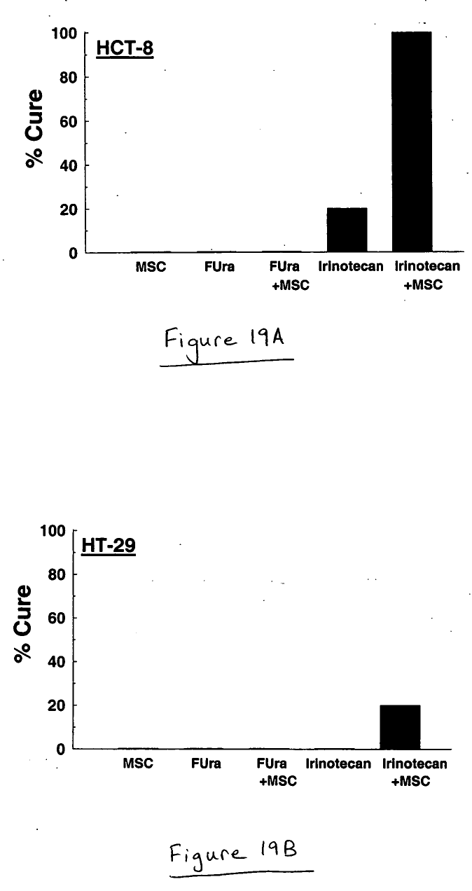

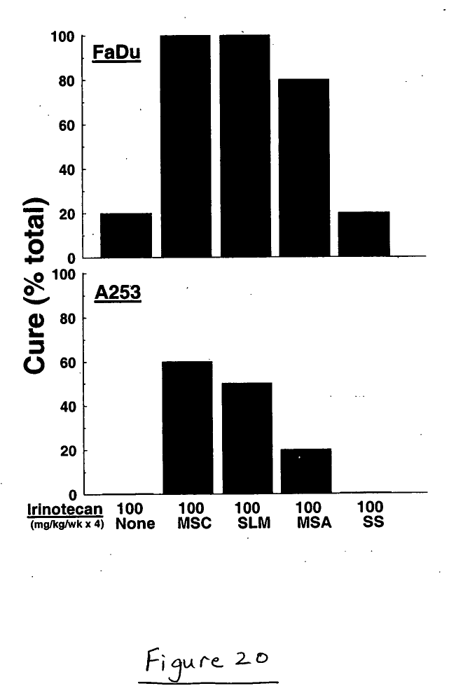

This example demonstrates that while selenium enhances the tumor volume reduction effect of FUra (FIGS. 18A and 18B), this effect is not durable and no effect on enhancing the percent cures was observed (FIGS. 19A and 19B). For these experiments, FUra was administered by i.v. push weekly times 4 and MSC by p.o. once a day for 6 weeks, the first dose being given 3 weeks before FUra administration. Irinotecan was administered by i.v. 100 mg/kg/week once a week for 4 weeks and MSC (0.2 mg/day), by p.o. daily for 28 days with the first dose started 7 days prior to irinotecan treatment.

In another experiment, several different chemotherapeutic agents were evaluated to determine if cure rates could be enhanced by the pre-administration of selenium. As shown in Tables 3 and 4, the augmentation of complete tumor regression activity by selenium was quantitatively drug specific. For example, while topotecan, campotothecin and irinotecan are topoisomerase I inhibitors, the complete tumor regression (cure) activity of only irinotecan was enhanced by selenium. Similarly, while taxol and taxotere are inhibitos of a microtubules, enhancement of complete tumor regression (cure) was observed only with taxotere but not with taxol.

TABLE 3

| | | | A253 | |
|---|---|---|---|---|
| | FaUra | | | PR |
| Treatment | CR (%) | PR (%) | CR (%) | (%) |
| FUra(100) | 0 | o | 0 | 0 |
| FUra(100) + MSC(0.2) | 0 | o | 0 | 0 |
| Xeloda(600) | 0 | o | 0 | 0 |
| Xeloda(600) + MSC(0.2) | 0 | o | 0 | 0 |
| Irinotecan(100) | 30 | 50 | 10 | 30 |
| Irinotecan(100) + MSC(0.2) | 100 | 0 | 60 | 40 |
| Topotecan(7.5) | 0 | 0 | 0 | 0 |
| Topotecan(7.5) + MSC(0.2) | 0 | 0 | 0 | 0 |
| Cyclophosphamide(100) | 0 | 0 | 0 | 0 |
| Cyclophosphamide(100) + MSC(0.2) | 0 | 0 | 0 | 0 |
| Doxorubicin(10) | 0 | 0 | 0 | 60 |
| Doxorubicin(10) + MSC(0.2) | 0 | 0 | 0 | 80 |
| Taxol(35) | 0 | 0 | 0 | 0 |
| Taxol(35) + MSC(0.2) | 0 | 0 | 0 | 20 |
| Taxotere(60) | 0 | 40 | 0 | 20 |
| Taxotere(60) + MSC(0.2) | 80 | 20 | 60 | 40 |
| Cisplatin(8) | 0 | 0 | 0 | 0 |
| Cisplatin(8) + MSC(0.2) | 0 | 0 | 0 | 0 |
| Oxaliplatin(15, i.v. × 1) | 0 | 0 | 0 | 0 |
| Oxaliplatin(15) + MSC(0.2) | 0 | 0 | 0 | 0 |
| Oxaliplatin(7.5, weekly × 4) | 0 | 0 | 0 | 0 |
| Oxaliplatin(7.5) + MSC(0.2) | 0 | 0 | 0 | 0 |

CR: complete tumor regression;
PR: partial tumor regression (>50% tumor reduction).
FUra, irinotecan, and oxaliplation (7.5 mg/kg) were administered by i.v. push once a week for 4 weeks (on day 0, 7, 14, and 21); topotecan cyclophosmide, doxorubicin, taxol, taxotere, cisplatin and oxliplatin (15 mg/kg) by a single i.v. injection and xeloda by p.o. daily for 7 days.
MSC (0.2 mg/mouse/day) was administered by p.o. daily for 14 days with single dose of drugs and 28 days with drugs given by weekly × 4 and started 7 days before chemotherapy. The tumors were transplanted 7 days before therapy.

TABLE 4

| | HCT-8 | | HT-29 | |
|---|---|---|---|---|
| Treatment | CR (%) | PR (%) | CR (%) | PR (%) |
| FUra(100) | 0 | 0 | 0 | 0 |
| FUra(100) + MSC(0.2) | 0 | 0 | 0 | 0 |
| Irinotecan(100) | 20 | 20 | 0 | 0 |
| Irinotecan(100) + MSC(0.2) | 100 | 0 | 20 | 0 |

CR: complete tumor regression;
PR: partial tumor regression (>50% tumor reduction).
FUra (100 mg/kg/week) and irinotecan (100 mg/kg/week) were administered by i.v. push once a week for 4 weeks and MSC (0.2 mg/day/mouse) by p.o. daily for 42 days with the first dose started 21 days before FUra or irinotecan. The tumors were transplanted 7 days before therapy.

### EXAMPLE 14

This example demonstrates that the antitumor activity of selenium is observed only with specific selenium compounds. It was observed that SLM and MSC (at 0.5 mg/day/rat daily for 14 days, the first dose being started at 7 days prior to doxorubicin) were effective in reducing toxicity, but not sodium selenite (data not shown). In this example, irinotecan was administered by i.v. push 100 mg/kg/week once a week for 4 weeks and MSC (0.2 mg/day), SLM (0.2 mg/day), MAS (0.1 mg/day) or SS (0.2 mg/day) by p.o. daily for 28 days with the first dose started 7 days prior to irinotecan treatment. The tumors were transplanted 7 days before irinotecan therapy. As shown in FIG. 20, the enhancement of the cure percentage of irinotecan was observed with SLM and MSC, but not with MSA or SS. Thus, SS, which is widely used clinically, has limited antitumor enhancing activity when combined with irinotecan.

These results indicate that selenium compounds, MSC and SLM augment the activity of anticancer agents, irinotecan and taxotere with respect to complete tumor regression. Those skilled in the art will recognize that based upon the disclosure herein, minor modifications will be apparent to those skilled in the art. Such modifications are intended to be within the scope of this invention.

1. A method for inhibiting the growth of a tumor in an individual comprising the steps of
a) administering to the individual a selenium compound selected from the group consisting of seleno-L-methionine and methylselenocysteine; and
b) administering to the individual a therapeutically effective dose of an anti-cancer agent selected from the group consisting of irinotecan and taxotere,
wherein the tumor undergoes complete regression upon administration of the anticancer agent and the selenium compound.

2. The method of claim 1, wherein the anticancer agent is irinotecan.

3. The method of claim 1, wherein the anticancer agent is taxotere.

4. The method of claim 1, wherein the selenium compound is seleno-L-methionine.

5. The method of claim 1, wherein the selenium compound is methylselenocysteine.

6. The method of claim 1, wherein the selenium compound is administered starting at least three days of prior to administration of the anticancer agent.

7. The method of claim 6, wherein the selenium compound is administered starting at least seven days of prior to administration of the anticancer agent.

8. The method of claim 1, wherein the tumor is selected from the group consisting of adenocarcinomas, melanomas, lymphomas, sarcomas, lung, breast, ovarian, head, neck, prostate, cervical, endometrial, colorectal, gastric, liver, fallopian tubes, esophagus, small intestine, pancreas, kidney, adrenal, vaginal, vulvar, brain and testes.

9. The method of claim 1, wherein the individual is a human.

10. The method of claim 9, wherein the administration of selenium results in serum selenium levels of at least about 1200 ng/ml.

11. The method of claim 10, wherein the tumor has proven to be refractory to an anticancer agent alone.

12. The method of claim 10, wherein the dose of selenium is at least 2.2 mg/day.

13. The method of claim 10, wherein the selenium is administered at a dose of between 3.2 to 5.6 mg one week prior to administration of the anticancer agent and is then administered at a dose of 2.8 to 5.6 mg per day thereafter.
